(19) United States
(12) Patent Application Publication
(10) Pub. No.: US 2022/0080096 A1
(43) Pub. Date: Mar. 17, 2022

(54) ION REDUCTION IN A BODY FLUID BY ZEOLITE

(71) Applicant: PREVIPHARMA CONSULTING GMBH, Mannheim (DE)

(72) Inventors: Maurice MANDAGO, Schoenau (DE); Stephan T. KIESSIG, Wiesloch (DE); Dirk MUELLER, Mannheim (DE); Ricarda WELZ, Mauer (DE)

(21) Appl. No.: 17/425,393

(22) PCT Filed: Jan. 23, 2020
(86) PCT No.: PCT/EP2020/051594

§ 371 Date: Jul. 23, 2021

(30) Foreign Application Priority Data
Jan. 24, 2019 (EP) 19153569.9

(51) Int. Cl. A61M 1/36; A61M 1/38

(52) U.S. Cl. CPC A61M 1/3672; A61M 1/38

(57) ABSTRACT

Surprisingly, it was found that zeolites can be used to decrease the concentration of inorganic ions, such as calcium ions, from a body fluid, such as plasma, in sufficient way. Such decrease of the concentration of inorganic ions, such as calcium ions, was found to lead to an inhibition of hemostasis including an inhibition of coagulation, especially inhibiting the activation of factor VII and the formation of fibrin.

Surprisingly, it was found that zeolites can be used to decrease the concentration of inorganic ions, such as calcium ions, from a body fluid, such as plasma, in sufficient way. Such decrease of the concentration of inorganic ions, such as calcium ions, was found to lead to an inhibition of hemostasis including an inhibition of coagulation, especially inhibiting the activation of factor VII and the formation of fibrin.

Body fluids often contain significant amounts of inorganic ions, including mono-, bi- tri- and polyvalent ions. While these have beneficial or even essential functions in an animal or human body, it is often desired to reduce certain ions, in particular bi- and/or trivalent ions, to a lower concentration level in samples of such body fluids withdrawn from a body. Reduction of the level of ions, in particular bi- and trivalent ions, may be beneficial for in vitro storability and usability of a body fluid.

For example, blood or blood plasma samples bear a tendency to coagulate (clot). This effect is, inter alia, promoted by the presence of calcium ions that are known to serve as a co-factor for coagulation. Therefore, for example, reduction of calcium and/or zinc levels in a blood or blood plasma sample may be desired to reduce the undesired coagulation (clotting) of the blood or blood plasma and/or to reduce enzymatic activities.

Reduction of bi- and trivalent ions such as, e.g., calcium and/or zinc levels, in a body fluid, such as blood or blood plasma, is often achieved by means of addition of complexing agents. One example of such complexing agents is citrate. Often, citrate is used for anticoagulation in collected blood or blood plasma samples by capturing free calcium ions causing hemostatic events and maintaining blood or blood plasma quality. The reduction of free calcium levels down to a concentration of 0.2-0.3 mmol/L causes a desired impairment of hemostasis. Although citrate is needed, lower levels of citrate (˜6%) result into higher yields of coagulation factors than higher citrate concentrations (˜8%).

Due to varying levels of citrate, caused by fluctuations in the volumes of the collected blood or blood plasma the citrate concentration might fall below the needed 15-24 mmol/L for a sufficient reduction of calcium. On the other hand, higher concentrations of citrate may have undesired toxic side effects when the stored blood or blood plasma is returned to a patient's body (Lee et al., J Clin Apher., 2012, 27(3):117-125; Bialkowski et al., Clin Apher., 2016, 31(5):459-463).

The concentration of the anticoagulant citrate is often reduced by the blood or blood plasma only and not with the amount of the cellular components of the blood. Therefore, the final concentrate of citrate may depend on the donors hematocrit (HCT) (which is a measure of the amount of cellular components in whole blood) and therefore highly individual. This results in one part of donors with a low hematocrit in an under-anticoagulation. The citrate concentration is diluted in high blood or blood plasma which results in a low citrate concentration. In the other part of donors with a high hematocrit in the opposite result is found with therefore an unnecessary higher dilution of plasma proteins. Undercoagulation may be a severe issue. This may be caused by an excess of added complexing agent over ions which can be complexed.

Due to in incomplete blocking of calcium ions, the coagulation based on a surface activation at plastic material of a device, such as apheresis sets, may be initiated. The fact that many of the coagulation factors are proteases and these proteases can digest other plasma proteins as well may be an issue. Therefore, the yield and the stability of many plasma proteins may further decrease. When returned to a patient's body, the calcium level is typically restored. This is often hampered by an excess of citrate in the blood or blood plasma sample.

In summary, high concentrations of complexing agents, such as citrate, often increases adverse events during the processing (including plasmapheresis/apheresis) and storage of a body fluid, such as blood and blood plasma samples. Therefore, it is desired to reduce the concentration of one or more species of inorganic ions in a body fluid without the need of high concentrations of complexing agents.

Other means known in the art, such as porous materials, such as zeolites, have other drawbacks. Such porous materials can remove calcium ions from liquid solutions and can be easily and efficiently removed from the liquid.

However, such materials having a rough surface, such as zeolites, increase coagulation via the intrinsic surface activation pathway. Accordingly, such materials having rough surfaces, such as zeolites, can be used as coagulation-promoting agents (Alam et al., Military Medicine, 2005, 170:63-69; Li et al., Acta Pharmacologica Sinica, 2013, 34:367-372). When, however, coagulation is not desired such as, e.g., in a stored blood or blood plasma sample, the usability of zeolites is hampered due to its tendency to promote undesired coagulation.

EP-A 0064393 teaches a dialysis method comprising an optionally calcium-loaded zeolite ion exchanger to remove uremic substances. EP-A 0046971 refers to a hemodialysis composition comprising a zeolite and exchangeable calcium load. Thus, the zeolite compositions of EP-A 0064393 and EP-A 0046971 may even increase calcium concentration of the treated body fluid.

In view of the above, there is an unmet need for means for reducing the concentration of one or more species of inorganic ions in a body fluid.

Surprisingly, it has been found that reducing the concentration of one or more species of inorganic ions in a body fluid can be obtained when using an ion-adsorbing zeolite separated from the body fluid by means of a semipermeable layer that allows the ions to pass through but not polypeptides. This can surprisingly minimize the amount of complexing agents, such as citrate, needed for anticoagulation or even replace the complexing agents. The quality of stored blood and blood plasma was found to be improved. Furthermore, it was surprisingly found that the method of the present invention can also be used for detoxification upon exposure to and uptake of toxic ions.

Accordingly, a first aspect of the present invention relates to a method for reducing the concentration of one or more species of inorganic ions in a body fluid, wherein said method comprises the following steps:
(i) providing:
   (A) the body fluid,
   (B) a semipermeable layer, which is permeable for the one or more species of inorganic ions, but which is (essentially) impermeable for polypeptides having a molecular weight of more than 40 kDa, in particular more than 10 kDa, and
   (C) a zeolite suitable for adsorbing the inorganic ions which is it is suspended in water or an aqueous buffer;
(ii) placing the semipermeable layer between the body fluid and the zeolite so that the body fluid does not get in direct contact with the zeolite; and
(iii) incubating the arrangement obtained from step (ii) under conditions that allow migration of the one or more species of inorganic ions through the membrane and adsorption thereof to the zeolite.

In a preferred embodiment, the method is an in vitro method, i.e., a method conducted outside the human or animal body.

A body fluid may be provided by any means. It may be a stored body fluid or a body fluid freshly obtained from a human or animal body (donor). Storage may be any kind of storage such as, e.g., storage at room temperature (RT), storage in an cool environment (e.g. in a fridge at a temperature in the range of 1 to 10° C.), storage in the frozen state (e.g. in a freezer at a temperature in the range of −25 to −1° C. or −90° C. to −60° C. or in liquid nitrogen at around −196° C.), or storage of a freeze dried state at any temperature, preferably below 30° C. Accordingly, storage may be at a temperature range of 15° C. to 30° C., 1° C. to 10° C., −25° C. to 25° C., −70° C. to −15° C., −90° C. to −60° C., −200° C. to −80° C., at around −196° C. or below −196° C. Storage may be storage for at least one or more minutes (min), for at least an hour (h), for at least six hours, for at least twelve hours, for at least a day (d), for at least a week, for at least a month, for at least two months, for at least six months, or for at least a year.

Provision of a body fluid freshly obtained from a human or animal body (donor) may be performed by any means. Preferably such provision is conducted by means which do not involve a (severe) health risk for the human or animal. For example, such provision may be blood sampling optionally followed by blood fractioning. Blood sampling may be, for instance venous blood sampling or arterial blood sampling.

The one or more species of inorganic ions may be any inorganic ions known in the art that may be comprised in a body fluid. The inorganic ions may be comprised in a body fluid naturally or may be a xenobiotic, including a toxic ion incorporated by intoxication. In a preferred embodiment, the inorganic ions in the sense of the present invention have an ionic weight of not more than 200 Da, in particular not more than 100 Da or not more than 50 Da. In a preferred embodiment, the inorganic ions in the sense of the present invention are one or more species of cations.

In a preferred embodiment, the inorganic ions are one or more species of cationic metal ions.

In the context of the present invention, an inorganic ion may have any valency. An inorganic ion may be a mono-, bi-, tri- or polyvalent inorganic ion. In a preferred embodiment, the one or more species of inorganic ions have a valency of 2 or more. In a preferred embodiment, the one or more species of inorganic ions are bi-, tri- or polyvalent inorganic ions. In a more preferred embodiment, the one or more species of inorganic ions are bi- or trivalent inorganic ions.

In a preferred embodiment, the inorganic ions in the sense of the present invention are one or more species selected from the group consisting of alkaline earth metals (group 2 of the periodic table of elements), earth metals (group 13 of the periodic table of elements, and a transition metal cation, each having a valency of II or III.

In a particularly preferred embodiment, the inorganic ions in the sense of the present invention are one or more species selected from the group consisting of calcium, zinc, magnesium, aluminum, lead, chrome, nickel, iron, cobalt, nickel, manganese, molybdenum, iridium, and copper ions.

In a particularly preferred embodiment, the inorganic ions in the sense of the present invention are one or more species naturally found in body fluid without xenobiotic influences.

In a particularly preferred embodiment, the inorganic ions in the sense of the present invention are one or more species selected from the group consisting of calcium, zinc, magnesium, aluminum, nickel, iron, and copper ions.

In a particularly preferred embodiment, the one or more species of inorganic ions comprise or are ions selected from the group consisting of calcium ions, zinc ions, and calcium and zinc ions. In a highly preferred embodiment, the one or more species of inorganic ions comprise or consist of calcium ions. In a preferred embodiment, the inorganic ions consist of calcium ions. In a preferred embodiment, the one or more species of inorganic ions comprise or are zinc ions.

In a preferred embodiment, the inorganic ions are zinc ions. In a preferred embodiment, the one or more species of inorganic ions comprise or consist of calcium and zinc ions. In a preferred embodiment, the inorganic ions consist of calcium and zinc ions.

In an alternative preferred embodiment, the inorganic ions in the sense of the present invention are one or more species of anions. In an alternative preferred embodiment, the inorganic ions in the sense of the present invention are one or more species of anions selected from the group consisting of carbonate (CO₃²⁻), and sulfate (SO₄²⁻).

The body fluid may be any type of body fluid. The body fluid may be a fluid from any animal species including humans. It may be obtained from a vertebrate (e.g., mammals including humans, birds, fishes, amphibia, reptiles, etc.) or from an invertebrate (e.g., arthropod (e.g., insects, spiders, crustace, crustaceans, etc.), mollusca, etc.).

In a preferred embodiment, the body fluid is obtained from a vertebrate or an insect. In a preferred embodiment, the body fluid is a vertebrate, in particular a mammal body fluid. In a preferred embodiment, the body fluid is a human body fluid.

In a preferred embodiment, the body fluid is selected from the group consisting of blood, blood plasma or a fraction thereof, and hemolymph or a fraction thereof. In a preferred embodiment, the body fluid is selected from the group consisting of mammal blood, mammal blood plasma or a fraction thereof. In a preferred embodiment, the body fluid is selected from the group consisting of human blood, human blood plasma or a fraction thereof. In a preferred embodiment, the body fluid is human blood. In a preferred embodiment, the body fluid is human blood plasma or a fraction thereof.

In a preferred embodiment, the body fluid is a unit of stored blood or plasma. In a preferred embodiment, the body fluid is a unit of mammal stored blood or plasma. In a preferred embodiment, the body fluid is a unit of human stored blood or plasma.

As indicated above, it is of particular interest to use the method of the present invention to at least partly inhibit coagulation and/or enzymatic activity of the body fluid.

In a preferred embodiment, the body fluid is a coagulatable body fluid. In a preferred embodiment, the body fluid is a coagulatable body fluid selected from the group consisting of mammal blood, mammal blood plasma, and a coagulatable fraction of mammal plasma. In a preferred embodiment, the body fluid is a coagulatable body fluid selected from the group consisting of human blood, human blood plasma, and a coagulatable fraction of human plasma.

As indicated above, the body fluid may or may not be subjected to further processes including apheresis. Accordingly, in a preferred embodiment, the body fluid is subjected to apheresis. In a preferred embodiment, the body fluid is selected from the group consisting of blood, blood plasma or a fraction thereof, and hemolymph or a fraction thereof subjected to apheresis. In a preferred embodiment, the body fluid is selected from the group consisting of mammal blood, mammal blood plasma or a fraction thereof subjected to apheresis. In a preferred embodiment, the body fluid is selected from the group consisting of human blood, human blood plasma or a fraction thereof subjected to apheresis.

The zeolite may be any zeolite in the art that can interact with the one or more species of inorganic ions of interest. In a preferred embodiment, the zeolite adsorbs at least parts of the one or more species of inorganic ions of interest. In a preferred embodiment, the zeolite is an aluminosilicate zeolite.

The zeolite may have any form. Typically, the zeolite is a solid that may be either a monolith or may be fragmented.

In a preferred embodiment, the zeolite is provided as a powder, in particulate form or as a paste.

In a preferred embodiment, the zeolite is provided as a powder. In a preferred embodiment, the zeolite is provided in particulate form. As used herein, the term “powder” may be understood in the broadest sense as any solid particles having a mean small diameter, such as <10 μm (e.g., determined by Laser diffraction analysis such as a Malvern Metasizer). As used herein, the term “in particulate form” may be understood in the broadest sense as any solid particles having a mean diameter between 0.01 mm and 10 mm, 0.1 to 5 mm, or 0.5 to 2 mm (e.g., determined by sieve analysis or microscopic or macroscopic measurements). A particulate form may have any shape. It may be (essentially) spherical, broken or crystalline. In a preferred embodiment, the zeolite particles are (essentially) spherical. In a preferred embodiment, the zeolite particles are (essentially) spherical and have a mean particle diameter of 0.1 to 5 mm (e.g., determined by sieve analysis or microscopic or macroscopic measurements).

In a preferred embodiment, the zeolite bears pores that may incorporate and at least partly capture the one or more species of inorganic ions of interest.

In a preferred embodiment, the zeolite has a pore size in the range of 140 to 600 pm. In a preferred embodiment, the zeolite has a pore size in the range of 300 to 500 pm. In a preferred embodiment, the zeolite has a pore size in the range of 350 to 450 pm, 375 to 425 pm, 380 to 420 pm, 390 to 410 pm or (around) 400 pm.

In a preferred embodiment, the zeolite is provided in a monovalent ion-loaded loaded form. In a preferred embodiment, the zeolite is provided in an alkaline metal loaded form such as, e.g., as sodium- and/or potassium-loaded zeolite.

It will be understood that the person skilled in the art may adapt pore size to the one or more species of inorganic ions of interest. In case the ions have a larger diameter, also the pore size may be chosen to be larger. In case the ions have a smaller diameter, also the pore size may be chosen to be smaller.

The zeolite may be used in a suspension or may be used in dry state. In a preferred embodiment, the zeolite is suspended in an aqueous buffer. As used throughout the present invention, the buffer is preferably a pharmaceutically acceptable buffer such as, e.g., phosphate buffered saline (PBS). As used herein, pharmaceutically acceptable may be understood in the broadest sense as any compound that can be used in a pharmaceutical context, i.e., is (essentially) non-toxic in the used concentration range. This can mean, but does not necessarily mean, that the buffer is officially approved for pharmaceutical uses (e.g., by the US and/or European Pharmacopeia, in particular each in the actual version at the filing date).

The zeolite may be suspended in an aqueous buffer prior to being used in the context of the present invention.

In a preferred embodiment, the zeolite is provided as a powder, in particulate form or as a paste and has a pore size in the range of 140 to 600 pm, in particular 300 to 500 pm. In a preferred embodiment, the zeolite is provided as a powder or in particulate form and has a pore size in the range of 300 to 500 pm.

As the zeolite preferably has a well-defined structure, the pore size is preferably well-defined. The pore size may also be designated as mean pore size. As used herein, the pore size may be determined by commons means in the art. As the zeolite preferably has a well-defined structure, the pore size may be directly obtained from the chemical structure. The pore size may refer to the hydrodynamic radius. In a preferred embodiment, the pores of the zeolite have (essentially) radial openings. In a preferred embodiment, the pores of the zeolite have (essentially) cylindrical pores. In case the pores of the zeolite do not have radial openings, the pore size preferably refers to the smallest diameter of the pore opening.

In another preferred embodiment, the pore size may also be determined as the specific surface area (SSA). Alternatively, also inverse size exclusion chromatography may be used for determining the pore size. Pore size may be determined as described in Chapter “Porosity and its Measurement” of Espinal in “Characterization of Materials”, edited by Elton N. Kaufmann, 2012, John Wiley & Sons, Inc.

In a preferred embodiment, the zeolite is provided as a powder, in particulate form or as a paste suspended in an aqueous buffer. In a preferred embodiment, the zeolite has a pore size in the range of 140 to 600 pm, in particular 300 to 500 pm and is suspended in an aqueous buffer. In a preferred embodiment, the zeolite is provided as a powder, in particulate form or as a paste suspended in an aqueous buffer and has a pore size in the range of 140 to 600 pm, in particular 300 to 500 pm.

The semipermeable layer is (essentially) impermeable for polypeptides having a molecular weight of more than 40 kDa (kilodaltons). As used herein, the term “essentially impermeable” in the context of polypeptides having a molecular weight of more than 40 kDa may be understood in the broadest sense in that polypeptides having a molecular weight of more than 40 kDa do not or nearly not pass through the semipermeable layer.

Accordingly, when a certain volume body fluid containing such polypeptides is subjected to one side of the semipermeable layer and a comparable volume of a buffer free of polypeptide is subjected to one side of the semipermeable layer, after incubation at room temperature for 24 hours, the buffer preferably does not contain more than 25% (mol/mol), more preferably does not contain more than 10% (mol/mol) or 5% (mol/mol), in particular does not contain more than 1% (mol/mol) of the concentration of polypeptides having a molecular weight of more than 40 kDa of the initial concentration of the body fluid.

In a preferred embodiment, the semipermeable layer is (essentially) impermeable for polypeptides having a molecular weight of more than 30 kDa, of more than 20 kDa, of more than 10 kDa, of more than 5 kDa, or of more than 2 kDa.

The semipermeable layer is permeable for the one or more species of inorganic ions. As used herein, the term “permeable” may be understood in the broadest sense in that such inorganic ions may efficiently pass through the semipermeable layer. Accordingly, when a certain volume body fluid containing such ions is subjected to one side of the semipermeable layer and a comparable volume of a buffer free of such ions, after incubation at room temperature for 24 hours, the buffer preferably contains more than 25% (mol/mol), more than 50% (mol/mol), or 75% (mol/mol), of the concentration of the respective one or more species of inorganic ions of the initial concentration of the body fluid.

In a preferred embodiment, the semipermeable layer has a cutoff for polypeptides in the range of below 1 kDa, below 2 kDa, below 5 kDa, below 10 kDa, below 20 kDa, below 30 kDa, below 40 kDa, or below 45 kDa. In a preferred embodiment, the semipermeable layer has a cutoff for polypeptides in the range of above 1 kDa, above 2 kDa, above 5 kDa, above 10 kDa, above 20 kDa, above 30 kDa, above 40 kDa, or above 45 kDa.

In a preferred embodiment, the semipermeable layer has a cutoff for polypeptides in the range of 2 to 45 kDa, 20 to 40 kDa, or 25 to 35 kDa. In a preferred embodiment, the semipermeable layer is an osmosis membrane, potentially even selective for certain ions. In a preferred embodiment, the semipermeable layer has a cutoff for polypeptides in the range of 2 to 45 kDa, 20 to 40 kDa, or 25 to 35 kDa and is an osmosis membrane, potentially even selective for certain ions.

A semipermeable layer may be a membrane or a more solid material. It may have any thickness suitable for the method of the present invention. It may, for example have a thickness in the range of 1 to 50 μm, 25 to 100 μm, 50 to 1000 μm, or 0.5 to 2 mm. The semipermeable layer may form part of a device usable in the context of the present invention.

In a preferred embodiment, the semipermeable layer forms part of a device selected from the group consisting of:

a filter, in particular a filter selected from the group consisting of a centrifugation filter, a filter for dead-end filtration under standard pressure, high pressure or vacuum, and a cross-flow filtration filter;
a dialysis device, in particular a dialysis device selected from the group consisting of a dialysis membrane, a dialysis tube, and a dialysis bag; and a hollow fiber.

Such devices are commercially available. The method may be conducted in an on-line procedure, wherein the body fluid passes by the semipermeable layer and, thus, the zeolite or may be conducted in a dead-end (i.e., batch) procedure wherein a batch of the body fluid is contacted with the semipermeable layer and, thus, the zeolite, for a defined time.

In a preferred embodiment, the method is conducted in an on-line (also designatable as in-line) procedure, preferably wherein the body fluid flows alongside the semipermeable layer, in particular wherein the method is conducted on-line within an apheresis or blood donation procedure.

In a preferred embodiment, in particular when conducted as an on-line procedure, step (iii) of incubating is conducted for at least 1 s (second), at least 10 s, or at least 20 s. In a preferred embodiment, in particular when conducted as a dead-end (i.e., batch) procedure, step (iii) of incubating is conducted for at least 5 min, at least 10 min, or at least 20 min.

The person skilled in the art may adapt the procedure to obtain a decrease of the concentration of the one or more species of inorganic ions of interest.

In a preferred embodiment, the step (iii) of incubating is conducted until the concentration of the one or more species of inorganic ions in the body fluid is reduced by at least 25% (mol/mol) in comparison to the concentration contained in the body fluid before conducting said method.

In a preferred embodiment, the step (iii) of incubating is conducted until the concentration of the one or more species of inorganic ions in the body fluid is reduced by at least 50% (mol/mol) or at least 75% (mol/mol) in comparison to the concentration contained in the body fluid before conducting said method.

The method of the present invention may be conducted as the sole method for reducing the concentration of one or more species of inorganic ions in a body fluid or may be combined with one or more further means usable for this purpose.

In a preferred embodiment, the method further comprises the addition of one or more complexing agents which complex the one or more of the one or more species of inorganic ions.

Such complexing agent may be any agent that is suitable for complexing the one more species of inorganic ions of interest.

In a preferred embodiment, the complexing agent is pharmaceutically acceptable. In a preferred embodiment, the complexing agent is a chelating agent. For example, the complexing agent may be selected from the group consisting of citrate and ethylenediaminetetraacetic acid (EDTA).

In a preferred embodiment, the method further comprises the addition of one or more agents which form precipitated with the one or more of the one or more species of inorganic ions. For example, higher concentrations of carbonates and/or phosphate ions may precipitate with cations, such as calcium cations.

As indicated above, the body fluid obtainable from the method of the present invention bears particularly beneficial properties. It has a reduced concentration of one or more species of inorganic ions of interest and does also not comprise large amounts of complexing agents.

Accordingly, a further aspect of the present invention relates to a body fluid having a reduced concentration of one or more species of inorganic ions, obtainable or obtained from a method of the present invention.

It will be understood that the definitions and preferred embodiments as laid out in the context of the method herein also mutatis mutandis apply to the obtainable body fluid. The method of the present invention may also be used to inhibit or prevent coagulation of a body fluid, wherein said method is conducted according to the present invention. Accordingly, a further aspect of the present invention refers to a method for preventing coagulation of a body fluid, wherein said method is conducted by reducing the concentration of one or more species of inorganic ions in a body fluid according to the present invention.

It will be understood that the definitions and preferred embodiments as laid out herein also mutatis mutandis apply to all methods of the present invention.

As indicated above, the method of the present invention may also be used for detoxification upon exposure to and uptake of toxic ions. Accordingly, a still further aspect of the present invention relates to a method for removing toxic inorganic ions from a body fluid, wherein said method is conducted according to the present invention.

Accordingly, an aspect of the present invention relates to a method for reducing the concentration of one or more species of inorganic ions in a body fluid, after intoxication, wherein said method is conducted in accordance with the present invention.

In a preferred embodiment, in this context, the one or more species of inorganic ions are one or more species of toxic inorganic ions such as, e.g., ions selected from the group consisting of lead, chrome, nickel, cobalt, nickel, manganese, molybdenum, and iridium ions. The person skilled in the art will, however, note that toxicity ma also depend on the incorporated amount. Thus, in principle, all ions, in particular inorganic bi-, tri- and polyvalent ions can lead to an intoxication. An aspect of the present invention relates to a method for reducing the concentration of one or more species of toxic inorganic ions in a body fluid (after intoxication of said one or more species of toxic inorganic ions), wherein said method is conducted in accordance with the present invention. In a preferred embodiment, the method further comprises using a dialysis procedure, in particular a peritoneal dialysis procedure for detoxification.

The following examples and figures are intended to provide illustrative embodiments of the present invention described and claimed herein. These examples are not intended to provide any limitation on the scope of the invented subject-matter. The invention is further illustrated by the figures, examples and claims.

### BRIEF DESCRIPTION OF THE FIGURES

FIG. 1 shows vials resulting from the thrombodynamics assay of a sample subjected to no treatment (FIG. 1A) in comparison to a sample subjected to 0.75 g/100 mL of a zeolite in a long-term incubation (FIG. 1B).

FIG. 2 shows vials resulting from the thrombodynamics assay of a sample subjected to no treatment (FIG. 2A) in comparison to a sample subjected to 0.75 g/100 mL of a zeolite in a short-term incubation (FIG. 2B).

FIG. 3 depicts a scheme of a device for removing calcium ions from human blood plasma by means of zeolites separated from human blood plasma by a semipermeable layer. Herein, an inner vial (1) containing a suspension of water or PBS buffer (2) with suspended zeolite beads (3) bears a ultrafiltration semipermeable layer (4). This inner vial (i) is placed into a main column (5), which contains a body fluid (6) (e.g., blood plasma).

FIG. 4 shows vials resulting from the thrombodynamics assay of a sample subjected to no treatment (FIG. 4A) in comparison to a sample subjected to a zeolite placed behind a semipermeable layer (FIG. 4B). The depicted results show thrombodynamics of blood plasma samples measured on the first day (upper row), and those stored for 7 days (middle row) and for 14 days (bottom row).

FIG. 5 shows factor VII levels after incubation of several days of a sample subjected to no treatment (black dots) in comparison to a sample subjected to a zeolite placed behind a semipermeable layer (white squares). The factor VII levels were determined by means of a chromatographic assay (FIG. 5A) and by means of a clotting assay (FIG. 5B).

### EXAMPLES

As noted above, calcium exists in the (human) blood in a concentration of approximately 2 to 2.5 mmol/L. Since the detection limit of calcium at classical analytical facilities is about 1.25 mmol/L (lower concentrations are below the detection level (bdl)), a condition, which can be fatal under normal circumstances, the pre-testing for the reduction capabilities of the zeolites was performed with higher calcium levels. Coagulation Factor VII (F.VII) was used as a highly sensitive marker for the influence of calcium levels onto the coagulation.

F.VII is a coagulation factor that is present at a low concentration and has a low resistance for being activated. A reduction of F.VII activation is a strong marker for an inhibited coagulation, thus, less product quality loss during plasma handling over the process of fractionation.

### Materials

Calcium chloride (CaCl₂)

Zeolite: Adsorbent UOP 4A-AP MolSiv Zeolite (Sodium Form), which is an aluminosilicate zeolite powder having a pore size of approximately 400 pm

Distilled water (H₂O dest.)

### Analytics

HPLC-FID

F.VII Clotting assay

F.VII chromogenic assay

F.VIIa chromogenic assay

### Preparation of Zeolites

The dry zeolite was hydrated with drops of H₂O dest. The zeolites were centrifuged before being used in the experiments.

### Example 1—Adsorption of Calcium Ions by Means of Zeolites in Artificial Solutions

Zeolites were tested for their ability to adsorb calcium ions from a solution with a CaCl₂ concentration of 10 mmol/L (approximately 5-fold of the plasma concentration). The activated hydrated zeolites were suspended in various concentrations and mixed with the CaCl₂ solution. The calcium concentration was measured after 30 min, 1 hour, 3 hours, 4 hours and 24 hours. The resulting calcium levels that were measured after 3 hours at five given concentrations of zeolite per 100 mL are depicted in Table 1 below.

TABLE 1
Resulting concentrations of calcium ions in the untreated
feed and after 3 hours of incubation at room temperature.

| Sample | Zeolite | Obtained calcium concentration |
|---|---|---|
| No. | concentration | [after 3 hours of incubation] |
| A0 | Feed (without zeolite) | 3.69 mmol/L |
| A1 | 6 g/100 mL | bdl (<1.25 mmol/L) |
| A2 | 3 g/100 mL, | bdl (<1.25 mmol/L) |
| A3 | 1.5 g/100 mL, | bdl (<1.25 mmol/L) |
| A4 | 0.75 g/100 mL | bdl (<1.25 mmol/L) |
| A5 | 0.375 g/100 mL | bdl (<1.25 mmol/L) |

Accordingly, the zeolites were able to effectively decrease the inorganic ions (calcium ions) at all tested concentrations, below the detection limit. The decrease of the calcium concentration in the feed may be due to adherence to the vessel walls.

### Example 2—Adsorption of Calcium Ions by Means of Zeolites in Human Blood Plasma

Three samples were tested with of plasma. The first sample (B0) consists of blood plasma without any zeolite. The second sample (B1) consists of blood plasma with 1.5 g/100 mL of the zeolite. The third sample (B2) contained sample B1 with a diminished calcium ion concentration to which a CaCl₂ solution has been added to re-calcificate the factors before the freezing. All samples were centrifuged before sampling to remove the zeolites. All three plasma samples were incubated for 24 hours at room temperature, then frozen and subsequently measured. The results are depicted in Table 2 below.

TABLE 2
Resulting concentrations of calcium ions in the blood plasma
measured after 24 hours of incubation at room temperature
with and without the addition of 1.5 g/100 mL of the zeolite.

| | | F.VII | | Ca²⁻ |
|---|---|---|---|---|
| Sample | | coagulation | chromogenic | concentration |
| No. | Description | [IU/dL] | [IU/dL] | [mmol/L] |
| B0 | untreated | 73.9 | 93.9 | 1.84 |
| | blood plasma | | | |
| B1 | blood plasma + | 120.4 | 118.3 | bdL |
| | zeolite | | | (<1.25 mmol/L) |
| B2 | blood plasma + | 130.9 | 51.4 | 3.08 |
| | zeolite + | | | |
| | re-calcification | | | |

These results demonstrate that untreated blood plasma (B0) showed lower values for in both assays, indicating a degradation of the factors. The addition of the zeolite (B1) effectively decreased the concentration of calcium ions. The subsequent addition of calcium ions (re-calcification, B2) restored the calcium ion concentration. The direct contact of zeolite with the blood plasma evidently triggered coagulation. This Example shows that decreasing calcium ion concentration by means of zeolites is also effective in blood plasma.

### Example 3—Adsorption of Calcium Ions by Means of Low Concentrations of Zeolites in Human Blood Plasma

Example 2 was repeated, while the concentration of the zeolite was decreased to 0.75 g/100 mL and the incubation time was extended to 48 hours at room temperature. The samples are analyzed after 24 hours and after 48 hours. The results are depicted in Table 3 below.

TABLE 3
Resulting concentrations of calcium ions in the blood plasma
measured after 24 hours and after 48 h of incubation at room
temperature with and without the addition of 0.75 g/100 mL of the zeolite.

| Sample | Time | | F.VII coagulation/ | Chromogenic |
|---|---|---|---|---|
| No. | [hours] | Description | Clotting [IU/dL] | [IU/dL] |
| C0 | 0 h | untreated | 66.2 | 66 |
| | | blood plasma | | |
| C0 | 24 h | untreated | 73 | 76 |
| | | blood plasma | | |
| C0 | 48 h | untreated | 70.3 | 80 |
| | | blood plasma | | |
| C1 | 24 h | untreated | 133.2 | 66 |
| | | blood plasma | | |
| C1 | 48 h | blood plasma + | 112.8 | 68 |
| | | zeolite | | |

### Example 4—Thrombodynamics Assay

### Introduction

The thrombodynamics assay (obtained from HemaCore) allows the visualization of real-time clot-formation and is one the assays with the closest comparability to the real coagulation in in vivo conditions. Samples were introduced in a chamber (cuvette), which was heated at 37° C. and calcium was added to the samples. The clotting was started by insertion of an activation which was covered with immobilized tissue factor, triggering the extrinsic coagulation pathway. The formation of the clot was measured by light scattering with red light at 625 nm. The clot formation was monitored with a CCD-cam and translated by the software into the following comparable variables:
T_{lag} [min]—Lag time, i.e. time between the contact of the activator with the plasma sample and the clot growth initiation;
V [μm/min]—average clot growth rate (from 15^{th} to 25^{th} min);
Tₛₚ [min]—time of spontaneous clot formation;
Vᵢ [μm/min]—average initial clot growth rate (from 2nd to 6th min). V=Vᵢ if spontaneous clotting does not allow normal V calculation;
D [a.u.]—clot density;
CS [μm]—size of the clot after 30 min (standard measurement length).
   The following experiments addressed the following questions:
How does the removal of calcium influence (inhibit) the coagulation?
Does the reduction of calcium reduce the amount of pre-activated coagulation factors (especially factor VII) over time (24 h) visible at the time for spontaneous clot formation in the assay?

### Example 5—Effect of Zeolites in Human Blood Plasma

Two samples of human plasma were thawed and zeolites were added to one of the samples in the concentration of 0.75 g/100 mL to reduce its calcium amount. Both samples were then tested after 2.5 h of incubation at room temperature (RT) by means of the thrombodynamic assay, but with no calcium and no inhibitor for the contact pathway added like in the standard protocol. The insertion of the immobilized tissue-factor was then used to start the coagulation, which is measured over a period of 1 hour.

The Test was performed in the absence of calcium ions. None of both samples (D1: non-treated; D2: calcium-reduced) showed the formation of any clotting during the assay.

### Example 6—Effect of Zeolites in Direct Long-Term Contact with Human Blood Plasma

Two samples of human plasma were thawed and zeolites were added to one of the samples in the concentration of 0.75 g/100 mL to reduce the concentration of calcium ions (sample E2). Another sample was not treated (sample E1). The samples from experiment one after at least 24 hours were measured by means of the thrombodynamics assay with the normal addition of calcium chloride in the standard assay procedure (see above).

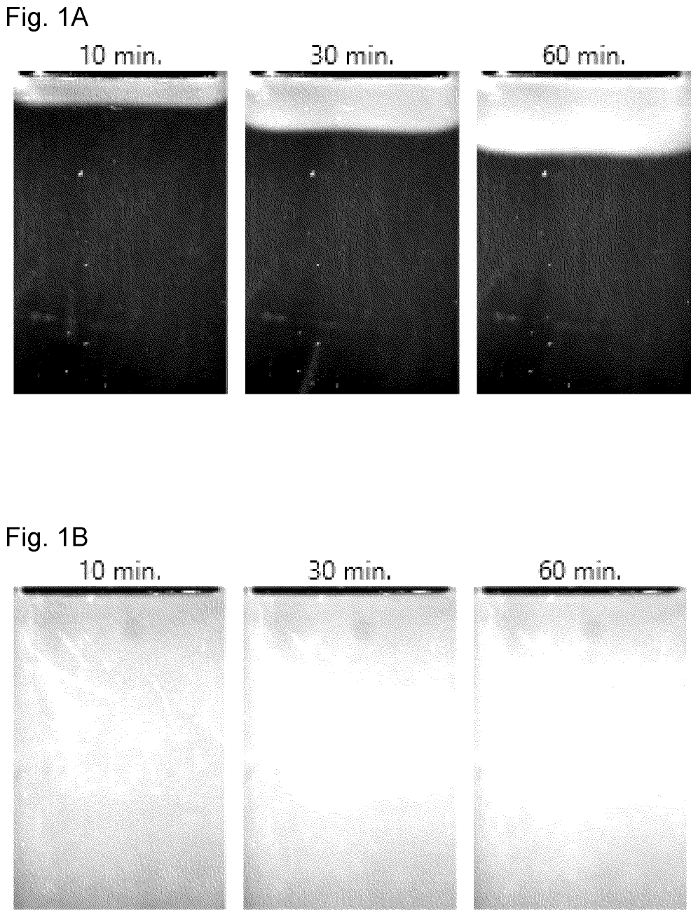

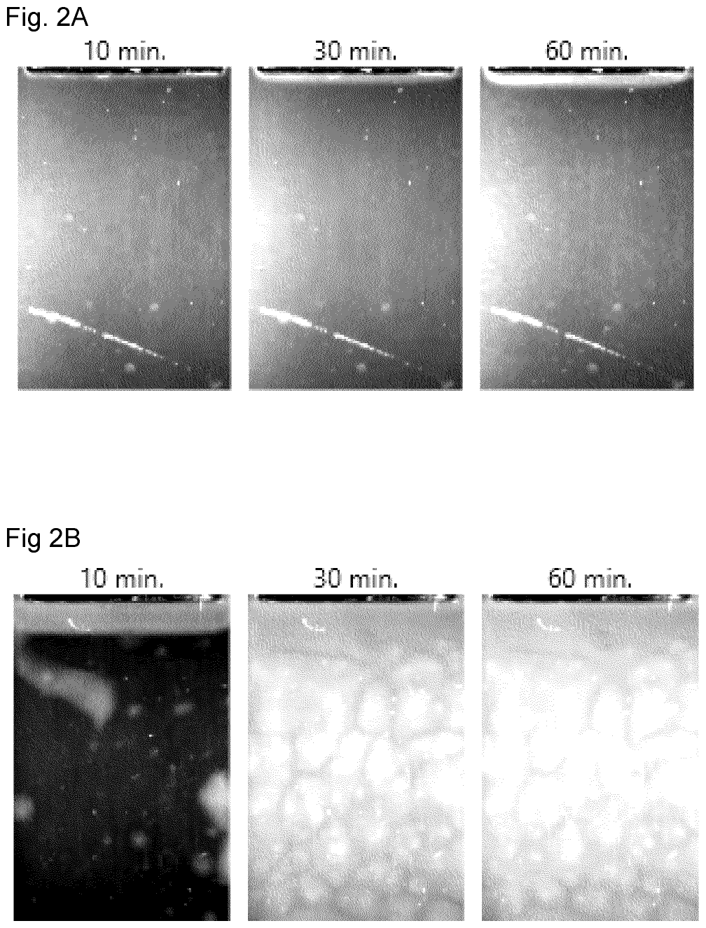

The calcium-reduced sample delivered a lower level of spontaneous activation and a longer T(sp) time. Results are also depicted in FIG. 1. This experiment delivered further unexpected results. The non-treated sample didn't show any formation of spontaneous clots, resulting in a non-existent Tₛₚ-value. The calcium-reduced sample showed significant levels of spontaneous clots, resulting in a low Tₛₚ value, therefore a quick formation of spontaneous clots.

TABLE 4
Resulting parameters found in blood plasma
after 24 hours of incubation at room temperature with
and without the addition of 0.75 g/100 mL of the zeolite.

| | | Sample E2 |
|---|---|---|
| | Sample E1 | (subjected to |
| | (untreated) | 0.75 g/100 mL) |
| T(Lag) [min] | 1.2 | — |
| V [μm/min] | 30.3 | — |
| T(sp) [min] | — | 5.9 |
| V(i) [μm/min] | 58.8 | — |
| D [a.u.] | 17813 | — |
| CS [μm] | 1285 | — |

Without being bound to this theory, it is assumed that the long contact with the surface of the zeolites, which weren't removed during the incubation, caused the activation of factor VII.

### Example 7—Effect of Zeolites in Direct Short-Term Contact with Human Blood Plasma

Example 6 was repeated, but instead of 24 hours, the samples E1 and E2 were merely incubated for 5 to 10 minutes and were then removed by centrifugation in order to prevent any further surface-related activation of factors. Sample E3 again showed the formation of spontaneous clots, indicating that the surface activation was not completely avoided. However, it was found to be reduced due to the reduction of contact time with the zeolites.

TABLE 5
Resulting parameters found in blood plasma after
5 to 10 min of incubation at room temperature with
and without the addition of 0.75 g/100 mL of the zeolite.

| | | E2 (subjected to |
|---|---|---|
| | E1 (untreated) | 0.75 g/100 mL) |
| T(Lag) [min] | 7 | 1.1 |
| V [μm/min] | 4.9 | 73.5 |
| T(sp) [min] | — | 11.6 |
| V(i) [μm/min] | 4.3 | 67.8 |
| D [a.u.] | 9768 | 14274 |
| CS [μm] | 156 | — |

Analysis of the calculated values showed that the spontaneous clot formation was significantly reduced, although not completely avoided. The T(sp) value changed from 5.9 min (cf. Experiment 6) to 11.6 minutes. The induced clot in sample E2 showed higher values for V and V(i), shorter value for T(lag) and a higher clot density D. The calcium level of the blood plasma was reduced from 3.69 mmol/L to 1.32 mmol/L. The calcium level was therefore still existent, but already in the range of a hypocalcemia. Likely, the measured calcium concentration was mainly composed of bound and non-ionized calcium, which is not involved in coagulation.

The samples were then tested with chromogenic assays which are less prone to erroneously high results from already activated factors, for their remaining levels of factor VII as an indicator for the long-term stability of the samples. The samples were measured after incubation at room temperature for 8 days.

After incubation at room temperature for 8 days, sample E1 showed a factor VII activation of 48.8 IU/dL, while sample E2 showed a factor VII activation of 58.2 IU/dL. Although sample E2 suffered from slight activation due to the calcium removal the remaining levels of factor VII were about 20% higher in comparison to its non-treated counterpart of sample E1. Calcium reduction seems therefore to be suited to prolong the half-life of labile coagulation factors, such as factor VII.

### Example 8—Effect of Zeolites in Direct Flow-Through Contact with Human Blood Plasma

In order to improve the efficacy of the calcium removal even further, a further approach was tried to decrease the undesired action (visible spontaneous clotting). Zeolites in the shape of small beads were packed into a chromatography column. Plasma was decalcified by flow-through through the activated zeolite column to have the shortest possible contact time. The proportional amount of zeolite per volume of plasma was increased and the contact time for the reaction drastically reduced to 1 minute.

### Materials and Methods

MiniVarioFlash® Flash-Kartuschen 2.5g (Götec)

NGC Quest HPLC System

### Results

The flow-through of the factors through the column resulted in strong pressure fluctuations, possibly due to the beads not being optimally packed. Finally, this experiment proved again the negative influence that the surface contact with the zeolites has on the coagulation factors leading to a strong visible activation.

### Example 9—Effect of Zeolites Separated from Human Blood Plasma by a Semipermeable Layer

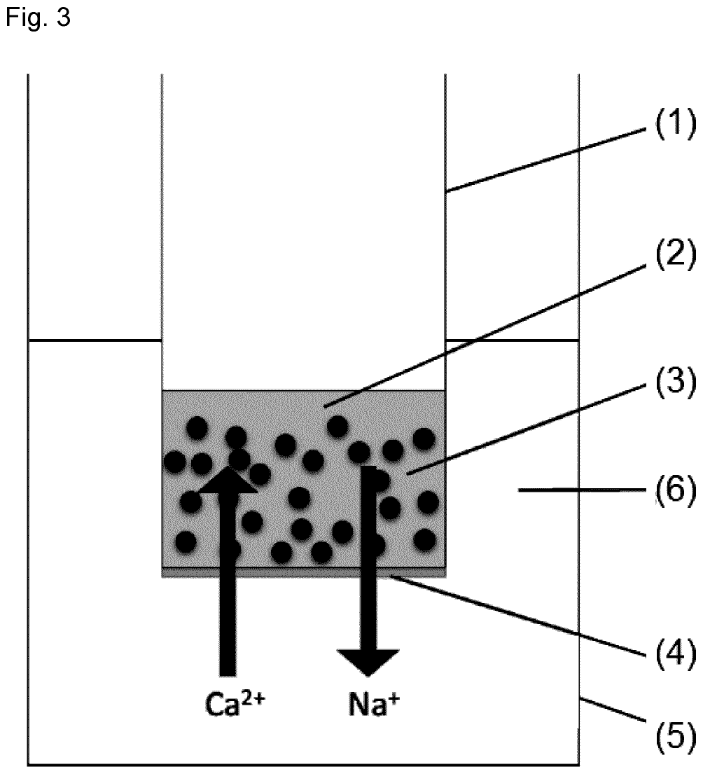

Since a direct contact with the zeolite resulted in an activation of coagulation factors, a system wherein the zeolites were separated from human blood plasma by a semipermeable layer was tested to remove the calcium from the plasma without any direct contact to the zeolites. The contact was limited by a semipermeable layer only allowing the diffusion of ions to reduce calcium levels in a fresh frozen and thawed plasma without affecting the quality of the sourced plasma. The setup is depicted in the scheme of FIG. 3.

### Materials

Zeolite Beads (4A MOLSIV®, Co. Obermeier)

CentriPrep YM-30

1×PBS buffer

water (H₂O dest.)

fresh frozen and thawed plasma

### Methods—Thrombodynamic, FVII Clotting and Chromogenic Assay

2 to 3 g (grams) of beads were filled into the inner chamber of the CentriPrep and water was added to activate them and incubated at room temperature for 1 hour. The water was decanted out of the inner chamber and PBS was filled in to neutralize the basic pH. 30 ml of plasma were thawed and 15 mL of plasma were filled into in the main column and the inner vial was inserted into the main column. The inner vial had contact with the plasma for 20 min before it was removed.

Performed Measurements:

Calcium: One measurement calcium removal (20 min)

Thrombodynamic Time intervals: 1 day, 1 week, 2 weeks

F.VII clotting Time intervals: 1 day, 1 week, 2 weeks, 3 weeks

F.VII chromogenic: Time intervals: 1 day, 1 week, 2 weeks, 3 weeks

### Results

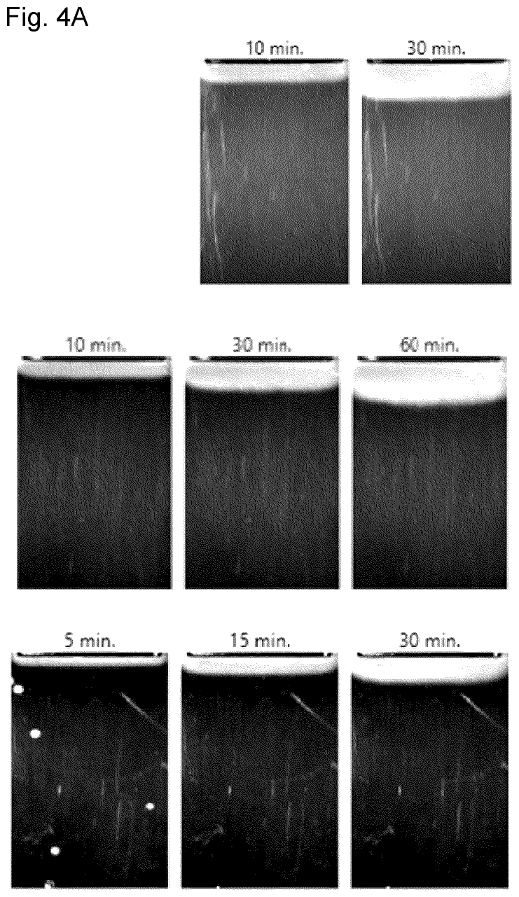

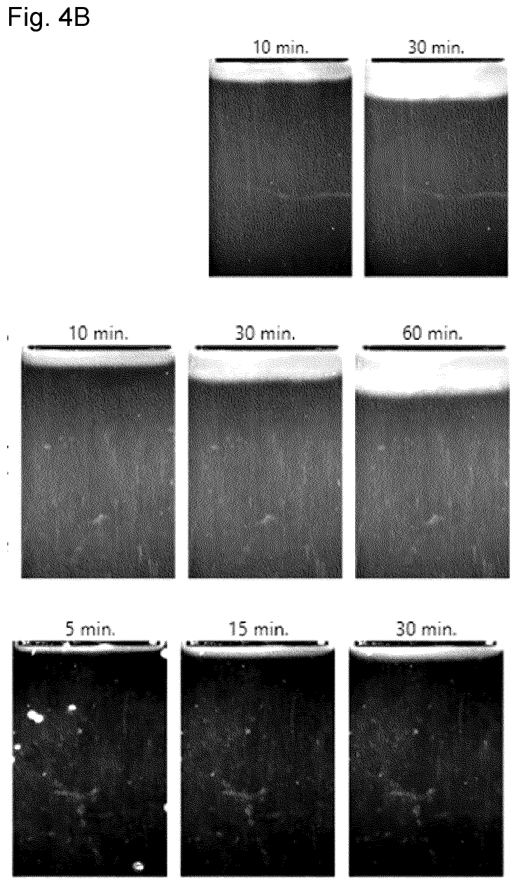

An untreated sample F1 was compared to a sample F2 that was subjected to calcium ion reduction by means of the zeolite placed behind a semipermeable layer. After 20 minutes the initial total calcium level (3.69 mmol/L) was reduced to 1.97 mmol/L. This implies an almost complete reduction of all free, non-bound calcium. The results regarding thrombodynamics (i.e., clotting) are depicted in FIG. 4. The thrombodynamics-assays of both samples resulted in the calculated values depicted in Table 6.

TABLE 6
Resulting parameters found in blood plasma of an untreated control
sample (sample F1) and a sample subjected to a zeolite by means of the
zeolite placed behind a semipermeable layer (sample F2).

| | Incubation of blood plasma | | | | | |
|---|---|---|---|---|---|---|
| | 0 days | | 7 days | | 14 days | |
| Sample | F1 | F2 | F1 | F2 | F1 | F2 |
| T(Lag) [min] | 1 | 1.2 | 1 | 1 | 1.2 | 1.7 |
| T(sp) [min] | — | — | — | — | — | — |
| V [μm/min] | 29.9 | 32.1 | 24.7 | 24.1 | 17.8 | 5.2 |
| V(st) [μm/min] | 29.9 | 32.1 | 24.7 | 24.1 | 17.8 | 5.2 |
| V(i) [μm/min] | 55.1 | 58.1 | 44.1 | 46.1 | 39.2 | 22.6 |
| D [a.u.] | 20353 | 18442 | 20965 | 16493 | 20133 | 14269 |
| CS [μm] | 1242 | 1295 | 1003 | 1001 | 793 | 384 |

Both, untreated control sample (F1) and calcium-reduced sample (F2) did not show any spontaneous clotting over all samples (i.e., T(sp) values were below detection limit). It was found that a semipermeable layer was very well useful to prevent undesired surface interaction using activation of factors, while maintaining the ability of the zeolite to reduce calcium concentrations.

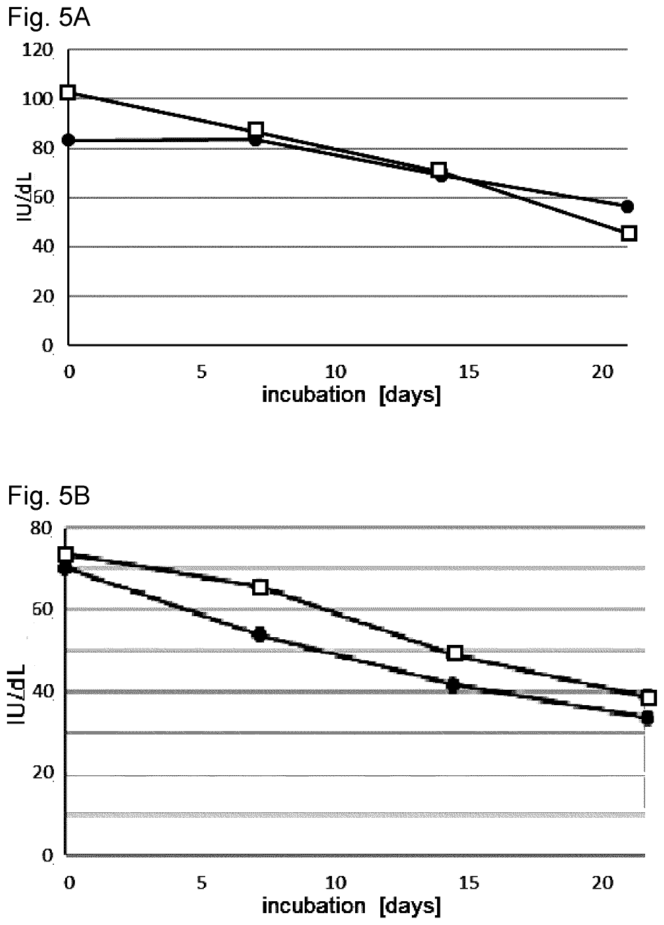

The factor VII levels were measured with both clotting and chromogenic assays. The factor VII levels were determined by means of a clotting assay (FIG. 5A) and by means of a chromatographic assays (FIG. 5B). The results are depicted in FIG. 5.

Both assays did not show a significant drop of factor levels during storage of the blood plasm samples F1 and F2 for 3 weeks. The levels of the calcium-reduced plasma were somewhat higher at the clotting assays, whereas no sample was significantly stronger at the chromogenic assay.

### DISCUSSION

The addition of a semipermeable layer in a device to remove calcium ions was found to be unexpectedly beneficial to prevent the surface contact inducing factor activation, thus damaging the plasma quality. Additional calcium reduction by zeolites was found to cause no undesired damage of already correctly decalcified plasma, thus making it a viable option for completion of the decalcification, e.g., in pooled plasma batches or to adjust the levels of unwanted ions, in particular bi- and trivalent ions, in body fluids.

1. A method for reducing the concentration of one or more species of inorganic ions in a body fluid, wherein said method comprises the following steps:
(i) providing:
(A) the body fluid,
(B) a semipermeable layer, which is permeable for the one or more species of inorganic ions, but which is essentially impermeable for polypeptides having a molecular weight of more than 40 kDa and
(C) a zeolite suitable for adsorbing the inorganic ions which is it is suspended in water or an aqueous buffer;
(ii) placing the semipermeable layer between the body fluid and the zeolite so that the body fluid does not get in direct contact with the zeolite; and
(iii) incubating the arrangement obtained from step (ii) under conditions that allow migration of the one or more species of inorganic ions through the membrane and adsorption thereof to the zeolite.

2. The method of claim 1, wherein the one or more species of inorganic ions are bi-, tri- or polyvalent inorganic ions, preferably wherein the one or more species of inorganic ions are bi- or trivalent inorganic ions, in particular wherein the one or more species of inorganic ions comprise or are ions selected from the group consisting of calcium ions, zinc ions, and calcium and zinc ions.

3. The method of claim 1, wherein the one or more species of inorganic ions comprise or consist of calcium ions.

4. The method of any of claims 1 to 3, wherein the body fluid is selected from the group consisting of blood, blood plasma or a fraction thereof, and hemolymph or a fraction thereof, in particular wherein the body fluid is a unit of stored blood or plasma.

5. The method of any of claims 1 to 4, wherein the body fluid is a coagulatable body fluid, in particular selected from the group consisting of mammal blood, mammal blood plasma, and a coagulatable fraction of mammal plasma.

6. The method of any of claims 1 to 5, wherein the body fluid is subjected to apheresis, in particular is selected from the group consisting of blood, blood plasma or a fraction thereof, and hemolymph or a fraction thereof subjected to apheresis.

7. The method of any of claims 1 to 6, wherein the zeolite is characterized by at least one of the following: it is provided as a powder, in particulate form or as a paste; it has a pore size in the range of 140 to 600 pm, in particular 300 to 500 pm; and/or it is suspended in an aqueous buffer.

8. The method of any of claims 1 to 7, wherein the semipermeable layer has a cutoff for polypeptides in the range of 2 to 45 kDa, 20 to 40 kDa, or 25 to 35 kDa and/or where the semipermeable layer is an osmosis membrane, potentially even selective for certain ions

9. The method of any of claims 1 to 8, wherein the semipermeable layer forms part of a device selected from the group consisting of a filter, in particular a filter selected from the group consisting of a centrifugation filter, a filter for dead-end filtration under standard pressure, high pressure or vacuum, and a cross-flow filtration filter; a dialysis device, in particular a dialysis device selected from the group consisting of a dialysis membrane, a dialysis tube, and a dialysis bag; and a hollow fiber.

10. The method of any of claims 1 to 9, wherein the method is conducted in an on-line procedure, preferably wherein the body fluid flows alongside the semipermeable layer, in particular wherein the method is conducted on-line within an apheresis or blood donation procedure.

11. The method of any of claims 1 to 10, wherein the step (iii) of incubating is conducted for at least 5 min, at least 10 min, or at least 20 min.

12. The method of any of claims 1 to 11, wherein the step (iii) of incubating is conducted until the concentration of the one or more species of inorganic ions in the body fluid is reduced by at least 25% (mol/mol) in comparison to the concentration contained in the body fluid before conducting said method.

13. The method of any of claims 1 to 12, wherein the method further comprises the addition of one or more complexing agents which complex the one or more of the one or more species of inorganic ions.

14. A body fluid having a reduced concentration of one or more species of inorganic ions, obtained from a method of any of claims 1 to 13.

15. A method for preventing coagulation of a body fluid, wherein said method is conducted by reducing the concentration of one or more species of inorganic ions in a body fluid according to any one of claims 1 to 13.

16. A method for reducing the concentration of one or more species of inorganic ions in a body fluid, after intoxication, wherein said method is conducted in accordance with any one of claims 1 to 13, preferably wherein said method further comprises using a dialysis procedure, in particular a peritoneal dialysis procedure for detoxification.
